(19) United States
(12) Patent Application Publication
(10) Pub. No.: US 2014/0275706 A1
(43) Pub. Date: Sep. 18, 2014

(54) SYSTEMS AND METHODS FOR DETERMINING AND DELIVERING RADIATION TREATMENT PLANS

(71) Applicant: Case Western Reserve University, Cleveland, OH (US)

(72) Inventors: David Dean, Cleveland, OH (US); Andrew Sloan, Cleveland, OH (US); Indraneel Gowdar, Cleveland, OH (US)

(21) Appl. No.: 14/212,094

(22) Filed: Mar. 14, 2014

Related U.S. Application Data
(60) Provisional application No. 61/798,169, filed on Mar. 15, 2013.
(60) Provisional application No. 61/802,874, filed on Mar. 18, 2013.

(51) Int. Cl. A61N 5/10

(52) U.S. Cl. CPC A61N 5/1031
USPC 600/1

(57) ABSTRACT

A computer-implemented method for generating and/or delivering a radiation treatment plan is provided. The method comprises reading a modular radiation dose kernel that defines a three dimensional radiation isocenter dose formed from a sum of a plurality of radiation beams, reading serial slice data that define three dimensional closed surfaces representing a lesion, critical structure, or organ and generating a radiation treatment plan based on the radiation kernel file and the data that defines the three dimensional closed surface. The treatment plan can be translated for use by virtually any automated radiation delivery device.

### RELATED APPLICATION

This application claims priority from U.S. Provisional Application Nos. 61/798,169, filed Mar. 15, 2013 and 61/802,874, filed Mar. 18, 2013, the subject matter of which are incorporated herein by reference in their entirety.

### TECHNICAL FIELD

The present invention relates to treatment planning and delivery, and particularly relates to systems and methods for generating and delivering radiation treatment plans including but not limited to delivering radiosurgery and radiation therapy.

### COPYRIGHT NOTICE

A portion of the disclosure of this patent document contains material subject to copyright protection. The copyright owner has no objection to the facsimile reproduction of the patent document or the patent disclosure as it appears in the Patent and Trademark Office patent file or records, but otherwise reserves all copyright rights whatsoever.

### BACKGROUND

Radiosurgery has typically been performed using a “step-and-shoot” approach that delivers radiation according to a three dimensional plan of multiple three dimensional radiation isocenter doses, also referred to as radiation “shots”. Multiple shots are usually required to destroy the target pathology. Step-and-shoot dose delivery involves the delivery of conformal radiation to the patient while the patient and radiation source are immobile, and then repositioning the patient and/or radiation source only when the patient is not exposed to the radiation source. This type of radiosurgery has been time consuming and may in some cases have produced sub-optimal results.

Radiosurgery may be performed using various devices. For example, Elekta (Stockholm, Sweden) provides a Leksell Gamma Knife™, which may be referred to as an LGK. The LGK provides accurate stereotactic radio surgical brain lesion treatment. The LGK derives its therapeutic radiation from 192 ⁶⁰Co radiation sources. A patient is exposed to these sources through collimator channels. The radiation beams passing through the collimator channels focus collectively in the center of a collimator helmet to create a conformal dose distribution. The dose drop-off is steep at the boundaries of this distribution (e.g., 90% to 20% isodose). The dose drop-off refers to how quickly the dose diminishes with distance from the center of the distribution. Since absolute doses vary depending on the duration of exposure, doses are typically expressed as a percentage of the maximum dose at the center of the distribution. However, dose drop-off steepness is diminished and made difficult to estimate when two or more shots have overlapping dose distributions.

Treatment planning seeks to achieve desired lesion coverage with the prescribed dose with multiple shots. However, “shot packing” is not as simple as filling a lesion, a theoretical bag, with ellipsoidal dose shots. Planning multiple shots is difficult due to the consequences of unintended intersections of beams from different shots. Although each of the beams comprising a shot are sub-lethal, beam crossings from multiple adjacent shots can accumulate significant dose in unintended areas. These unintended intersections of beams from different shots complicate treatment planning, and thus lengthen the time required to plan a multi-shot treatment.

Planning and delivery complexity are related to the geometric complexity and volumetric complexity of the target volume. For example, large lesion volume, complex lesion shape, and/or complicated geometric relationships between the lesion and critical structures complicate planning, and thus increase planning time and increase the likelihood that suboptimal results will occur.

Conventional LGK treatment planning begins with a treatment planning team that includes, for example, a neurosurgeon, a radiation oncologist, and a radiation physicist. The treatment planning team may survey pre-radiosurgical images (e.g., CT, MR) to locate the lesion in a series of adjacent 2D image slices. Drawing the boundary of the lesion or lesions is referred to as “segmentation”. Other objects of interest, (e.g., critical structures near the lesion), may also be segmented (i.e., identified) at this time. Segmentation is typically performed manually using a contour drawing tool. Shot packing strategies may not begin until the entire set of image slices is available.

Conventional treatment planning falls into two categories: forward treatment planning, and inverse treatment planning. Treatment planning begins with known parameters including prescribed dose, lesion location, segmented tissue object contours, and so on. Forward planning includes a trial-and-error approach for choosing shot parameters including number of shots, shot positions, collimator sizes, shot weights, and so on. As shot parameters are selected the treatment planning team can calculate and evaluate the sum of the radiation dose distribution. The treatment team will then manually adjust setup parameters until an “acceptable” treatment plan is obtained. This is an extremely technical and manual process requiring the input of several highly skilled personnel. This approach is not deterministic.

Given time limitations imposed by single session treatment a significant issue for forward treatment planning is the relative size of the search and solution space for acceptable treatment plans. The shots resulting from this trial-and-error procedure may produce unintended radiation dose overlap, particularly when multiple shots are placed in close proximity. However, the treatment plan search space increases dramatically when a lesion has a large target volume, a complex target shape, and/or a complex geometric relationship between the target volume and nearby critical section (CS). In this situation, treatment planning may require hours to obtain an acceptable treatment plan.

### SUMMARY

The present invention relates to systems and methods of radiation treatment planning and delivery. In some example embodiments, the subject invention is directed to an automated radiosurgery treatment planning and delivery of lethal radiation to brain lesions and other intra-cranial lesions. In other example embodiments, the present invention is directed to improvements on the previously patented Tomosurgery treatment planning and delivery algorithm (U.S. Pat. No. 7,616,735, the entire contents of which is hereby incorporated by reference herein) and the implementation of the Tomosurgery treatment planner in radiosurgery treatment systems such as the Leksell Gamma Knife® (LGK, Eleckta, Stockholm, Sweden), and/or the CyberKnife®* (Accuray, Sunnyvale, Calif.), and/or other commercial radiosurgery and/or radiotherapy treatment devices. Furthermore, the subject invention is related to several improvements of the use of these systems employing the various improvements in the software algorithms and implementations.

In some example embodiments, the improved Tomosurgery treatment planner is not limited to a co-planar beam arrangement (i.e., a disk-shaped shot in original algorithm), but can accommodate any feasible dose distribution isocenter (e.g., semi-spherical shaped shot). Previously, the planner was limited to a particular beam arrangement of a particular device (i.e., the disk-shaped shot using the LGK. In this embodiment, it can accommodate any dose distribution from any radiotherapy device capable of creating a dose isocenter (i.e. not limited to LGK).

In other example embodiments, the improved Tomosurgery treatment planner can be implemented with multiple sizes of shots (i.e., beams can have different sizes). In yet other embodiments, the improved Tomosurgery treatment planner is provided with a user interface that allows for User-defined dose inhomogeneity, which allows a user to adjust the intensity modulation. In further embodiments, a Dose engine with an associated user interface is provided for the Elekta Leksell Gamma Knife® Perfexion™. The dose engine allows for user-defined shapes, sizes and inhomogeneity in a user-defined dose kernel. The modularity allows for flexible shifting between devices. In yet other example embodiments, improvements on the Tomosurgery treatment planner include executing the Tomosurgery treatment planner with parallel processors to allow for the processing of multiple two-dimensional plans concurrently, as well as utilizing graphics processors (GPUs), greatly increasing the speed of the formation of the treatment plan.

In one example, a computer-implemented method is provided for generating a radiation treatment plan. The method comprises reading a radiation dose kernel that defines a three dimensional radiation dose isocenter formed from a sum of a plurality of radiation beams, reading data that defines a three dimensional closed surface representing a lesion, and generating a radiation treatment plan. The generating of the treatment plan comprises determining a three dimensional radiation isocenter dose from the loaded radiation dose kernel, logically dividing the three dimensional closed surface into a plurality of treatment slices, determining and optimizing an initial path plan for moving the three dimensional radiation isocenter dose through each treatment slice for each of the treatment slices, and determining and optimizing a final path plan for moving the three dimensional radiation isocenter dose through each treatment slice for each of the treatment slices to mitigate beam crossings effects that occur in treatment slices during the initial path plan.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates a system for providing radiation treatment in accordance with an embodiment of the present invention.

FIG. 2 illustrates an example dividing of a lesion image into multiple treatment slices and the raster dose delivery to a given slice by moving a disk-shaped shot over the given slice.

FIG. 3 illustrates a side schematic view of an Elekta Leksell Gamma Knife® Perfexion™.

FIG. 4 illustrates an example of a dose calculation interface for a dose engine configured to utilize modular dose kernel that can be employed to generate a radiation treatment plan for a Leksell Gamma Knife® Perfexion™ system.

FIG. 5 illustrates an example of skull measurements that are used for the dose calculation interface of FIG. 4.

FIGS. 6a-6b illustrate other examples configurations of the dose calculation interface of FIG. 4.

FIG. 7 illustrates an example embodiment of a method for generating a radiation treatment plan.

FIGS. 8a-8i illustrate a visual walkthrough of the GUI Software during execution of a treatment plan by the treatment planner.

FIG. 9 illustrates a user-defined dose inhomogeneity interface that allows for a user to provide a user-defined adjusted inhomogeneity dose in the treatment plan.

FIG. 10 illustrates a method for phase 1 slice optimization in accordance with one or more embodiments of the invention.

FIG. 11 illustrates a method for phase 2 plan optimization in accordance with one or more embodiments of the invention.

FIG. 12 is a schematic block diagram illustrating an example system of hardware components capable of implementing examples disclosed in FIGS. 1-11.

### DETAILED DESCRIPTION

Systems and methods are disclosed for determining and delivering a radiation treatment plan. These systems and methods employ a dose kernel reader to allow for the utilization of modular dose kernels for defining a three dimensional radiation isocenter dose (also referred to as radiation shots) formed from a sum of a plurality of radiation beams used in virtually any radiotherapy and/or radiosurgery device. The dose kernel reader can load one or more kernel files and/or parameters to produce hybrid radiation shots. The dose kernel reader allows for modular systems and methods that can provide for any of a variety of delivery schemes and virtually any radiotherapy or radiosurgery device. Furthermore, the dose kernel can be utilized to calibrate the radiation delivery device. The systems and method can also employ a lesion image data reader to allow for the utilization of different image data formats to define a three dimensional closed surface that represents a lesion. The systems and method can further include tools for skull measurements (surface approximation models) needed for dose calculation, such as but not limited to a bubble helmet skull model (e.g., 32 ref pts.). Additionally, tools are provided to calculate dose delivered by each beamlet.

In accordance with one or more examples, the systems and methods are instantiated for the LGK Perfexion, such that each pixel is instantiated as dose unit/min, which can be used to track dose at each target location. The systems and methods allow for the ability to quickly change duration of dose at each target location to insure optimality. In accordance with one or more examples, a deterministic dose calculation model has been instantiated that employs a convolution-superposition algorithm that improves accuracy compared to a Monte Carlo algorithm.

In accordance with one or more examples, at least a portion of the systems and methods can be instantiated to use GPU processing and/or parallel CPU processing to improve calculation times dramatically. These techniques can improve calculation times from hundred of seconds to less than ten seconds. GPU processing is faster than parallelized code and can be implemented via OpenCL/C99 (Khronos, Inc.) and optimized for massively-multithreaded GPU. The systems and methods provide techniques that have been instantiated to produce DICOM-RT files. Prototypes have been executed employing MatLab and ported to MS Visual Studio C# 2010.

Although some examples have been illustrated and instantiated on the GK Perfexion, it is appreciated that the techniques of the systems and methods can be applied to more radiation delivery machines readily through the plug-in of a modular dose kernel. Further examples provide instantiations of: test input of plans from external treatment planning software, tests 3D distribution (output) of executed plan using gel dosimeter, cases with and without adjacent critical structures, rapid drop off implemented hot spots, adaptation of shots to shot shapes of devices, maximization of plan optimality, a comparison of desired dose map and actual dose map, and a maximization of dose delivery speed.

FIG. 1 illustrates a system 10 for providing radiation treatment in accordance with an embodiment of the present invention. Although FIG. 1 is illustrated as a system 10, it is to be appreciated that at least a portion of the system 10 can be readily implemented as a methodology that employs computer executable components. It is further appreciated that the system can be one or more computer systems that execute the components of the system 10. The system 10 includes a dose kernel reader 14, a lesion image reader 16, a treatment planner 18, a graphical user interface (GUI) 20 coupled to the GUI 20 and a treatment deliver apparatus 22. The dose kernel reader 14 is configured, when executed, to read and load a dose kernel to the treatment delivery planner 18, and a lesion image reader 16 is configured, when executed, to read and load lesion image data to the treatment delivery planner 18. The treatment delivery planner 18 is configured to generate a radiation treatment plan to a treatment delivery apparatus 22 for performing radiotherapy or radiosurgery on a lesion of a patient utilizing the loaded dose kernel and the loaded lesion image data. A treatment planner graphical user interface (GUI) 20 is provided that allows a user to view and modify various parameters during different phases of the treatment planning process. The GUI 20 also allows a user to load different radiation kernel files and lesion image data files, and view various views and profiles of the radiation doses, radiation dose rates slice-by-slice of lesion images as will be described below.

The dose kernel can be provided by a dose engine 12 which provides an open architecture that allows a user to define the dose rate distribution of a single radiation shot at an isocenter. Alternatively, a modular dose kernel can be provided by, for example, a third party. The dose kernel can be a three dimensional ASCII matrix (e.g., 161×161×161) with a given voxel resolution (e.g., 0.25×0.25×0×25 mm) that collectively defines a three dimensional radiation shot size and shape. Different dose kernels can be employed based on different collimator sizes (e.g., 4 mm, 8 mm, 16 mm for LGK devices) and given voxel sizes (e.g., 0.25, 0.5 mm). Furthermore, asymmetric or irregularly shaped isocenters can be used, such as the hybrid isocenters of the LGK Perfexion. The lesion image data can be in a single DICOM (Digital Imaging and Communications in Medicine) volume image file, a series of DICOM slice images, or other slice image format where pixel size and slice thickness are known. The one or more DICOM image files can include one or more DICOM RT (Radiation Therapy) files that include, or will eventually include, user-defined regions of interests (ROIs) of lesions and/or critical structures defined as three dimensional closed surfaces (i.e., each object identified presents a single pixel or a closed contours in the slice image).

In one or more embodiments of the present invention, the treatment planner 18 is an improved Tomosurgery treatment planner based on Tomosurgery treatment planner disclosed in U.S. Pat. No. 7,616,735. Tomosurgery involves slice based radiosurgery treatment planning that utilizes a moving, high-precision, controlled-shaped isocenter between scan points along a set of scanning lines in a portion of a target volume divided into sets of treatment planes, referred to as slices. The resulting slice-based raster treatment plan can be, but need not be, delivered in the same way.

In one example, the scan points may be visited in a raster-scanning pattern controlled by a set of 2D plans. If it is optimal, raster scan points may also be, but are not required to be, visited slice-by-slice through a target volume as controlled by a 3D plan built from the set of 2D plans during radiation delivery. The slice thickness may be optimized to smooth peak-to-peak transitions between slices. Shot weight can be adjusted by controlling parameters including, but not limited to, shot movement speed, shot movement location, the number of beams being used in the shot, the distance of the radiation source from the target volume, and the size of beams used in the shot. In the case of the Leksell Gamma Knife Perfexion, the number of beams and the size of the beams may be determined by controlling on-the-fly collimator changes (e.g., sector arrangement and beam collimator size), and/or by controlling a radiation source position. The location of the isocenter can be moved by controlling parameters including, but not limited to, the number of different beams being used, temporal delays between beams, delivery apparatus location and/or orientation, radiation source location, and/or orientation, and patient location and/or orientation.

Example systems and methods may perform intensity modulated radiation therapy (IMRT) by modulating the speed of a moving shot or moving shots. Since the deposited dose depends on how long the isocenter lingers in a certain location, the overall dose can be modulated by changing the speed of the moving shot. IMRT may rely on achieving relative motion between a patient and a radiation field to provide a planned radiation dose in a continuous fashion. The relative motion may be achieved by moving the patient, by moving the delivery apparatus, by moving the radiation source, and by combinations thereof.

While some example systems and methods are described in association with an LGK, the systems and methods are not so limited. For example, treatment planning and radio-surgery may be associated with other delivery mechanisms and radiation sources including, for example, a cyberknife having a single point source of radiation, a C arm linear accelerator, an apparatus having multi-leaf collimators (MLC), and so on.

Similarly, while example systems and methods are described in connection with brain surgery, the systems and methods are not so limited. For example, treatment planning and radiosurgery may be applied to other body parts including, for example, the torso, extremities, and so on. Additionally, while the examples are described in terms of human treatment, radiosurgery may be performed on additional subjects (e.g., dogs, horses, cows).

Additionally, while example systems and methods describe a raster based approached associated with a moving shot, it is to be appreciated that other motion patterns may be employed. Raster based approaches may simplify mechanical adaptations to conventional apparatus and may facilitate simplifying motion plan computations. However, in some examples, other motion plans (e.g., helical, spiral) for the moving shot may be employed.

References to “one embodiment”, “an embodiment”, “some embodiments”, “one example”, “an example”, “some examples” and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

The following includes definitions of selected terms employed herein. The definitions include various examples and/or forms of components that fall within the scope of a term and that may be used for implementation. The examples are not intended to be limiting. Both singular and plural forms of terms may be within the definitions.

“Machine-readable medium”, as used herein, refers to a medium that participates in directly or indirectly providing signals, instructions and/or data that can be read by a machine (e.g., computer). A machine-readable medium may take forms, including, but not limited to, non-volatile media (e.g., optical disk, magnetic disk), and volatile media (e.g., semiconductor memory, dynamic memory). Common forms of machine-readable mediums include floppy disks, hard disks, magnetic tapes, RAM (Random Access Memory), ROM (Read Only Memory), CD-ROM (Compact Disk ROM), and so on.

“Data store”, as used herein, refers to a physical and/or logical entity that can store data. A data store may be, for example, a database, a table, a file, a list, a queue, a heap, a memory, a register, a disk, and so on. In different examples a data store may reside in one logical and/or physical entity and/or may be distributed between multiple logical and/or physical entities.

“Logic”, as used herein, includes but is not limited to hardware, firmware, executing instructions, and/or combinations thereof to perform a function(s) or an action(s), and/or to cause a function or action from another logic, method, and/or system. Logic may include a software controlled microprocessor, discrete logic (e.g., application specific integrated circuit (ASIC)), an analog circuit, a digital circuit, a programmed logic device, a memory device containing instructions, and so on. Logic may include a gate(s), a combinations of gates, other circuit components, and so on. Where multiple logical logics are described, it may be possible in some examples to incorporate the multiple logical logics into one physical logic. Similarly, where a single logical logic is described, it may be possible in some examples to distribute that single logical logic between multiple physical logics.

An “operable connection”, or a connection by which entities are “operably connected”, is one in which signals, physical communications, and/or logical communications may be sent and/or received. An operable connection may include a physical interface, an electrical interface, and/or a data interface. An operable connection may include differing combinations of interfaces and/or connections sufficient to allow operable control. For example, two entities can be operably connected to communicate signals to each other directly or through one or more intermediate entities (e.g., processor, operating system, logic, software). Logical and/or physical communication channels can be used to create an operable connection.

“Signal”, as used herein, includes but is not limited to, electrical signals, optical signals, analog signals, digital signals, data, computer instructions, processor instructions, messages, a bit, a bit stream, or other means that can be received, transmitted and/or detected.

“Software”, as used herein, includes but is not limited to, one or more executing computer instructions that temporarily transform a general purpose machine into a special purpose machine. Software causes a computer, processor, or other electronic device to perform functions, actions and/or behave in a desired manner. Software may be embodied in various forms including routines, algorithms, modules, methods, threads, and/or programs. In different examples, software may be implemented in executable and/or loadable forms including, but not limited to, a stand-alone program, an object, a function (local and/or remote), a servelet, an applet, instructions stored in a memory, part of an operating system, and so on. In different examples, computer-readable and/or executable instructions may be located in one logic and/or distributed between multiple communicating, co-operating, and/or parallel processing logics and thus may be loaded and/or executed in serial, parallel, massively parallel and other manners.

“User”, as used herein, includes but is not limited to, one or more persons, software, computers or other devices, or combinations of these. Some portions of the detailed descriptions that follow are presented in terms of algorithm descriptions and representations of operations on electrical and/or magnetic signals capable of being stored, transferred, combined, compared, and otherwise manipulated in hardware. These are used by those skilled in the art to convey the substance of their work to others. An algorithm is here, and generally, conceived to be a sequence of operations that produce a result. The operations may include physical manipulations of physical quantities. The manipulations may produce a transitory physical change like that in an electromagnetic transmission signal.

It has proven convenient at times, principally for reasons of common usage, to refer to these electrical and/or magnetic signals as bits, values, elements, symbols, characters, terms, numbers, and so on. These and similar terms are associated with appropriate physical quantities and are merely convenient labels applied to these quantities. Unless specifically stated otherwise, it is appreciated that throughout the description, terms including processing, computing, calculating, determining, displaying, automatically performing an action, and so on, refer to actions and processes of a computer system, logic, processor, or similar electronic device that manipulates and transforms data represented as physical (electric, electronic, magnetic) quantities.

FIG. 2 illustrates an example dividing of a lesion image into multiple treatment slices and the raster scanning of a given slice by moving a disk-shaped shot over the given slice. In the Tomosurgery treatment planner in general, the lesion volume is divided into N slices (as illustrated in FIG. 2(a)) with each slice being treated independently from the first slice, where N is a positive integer greater than one. The dose kernel is moved along a raster-like path within the treatment slice, “painting” the dose to fill the entire slice as illustrated in FIG. 2(b). A cost-function optimizes the path and speed. Next, the 2D treatment plans are assembled and optimized to account for doses from previous slices and critical structures to provide a determined radiation treatment plan. In the improved implementation of the Tomosurgery treatment planner, the radiation shot is not limited to a disk-shaped shot since the dose kernel is independent of the Tomosurgery treatment planner and can define a variety of different radiation shot shapes, sizes and distributions.

FIG. 3 illustrates a side schematic view of an Elekta Leksell Gamma Knife® Perfexion™ 30, which is the newest iteration of Gamma Knife treatment delivery apparatus technology. The Elekta Leksell Gamma Knife® Perfexion™ employs 192 ⁶⁰Co sources arranged in a cylindrical configuration in five concentric rings with eight sectors of 24 beams per sector. The Elekta Leksell Gamma Knife® Perfexion™ allows for three possible approaches in the treatment planning: 1) use of classical combinations of 4, 8, and 16 mm isocenters (shots), 2) use of composite shots containing combinations of 4, 8, and 16 mm or blocked sectors and 3) dynamic shaping using blocked selected sectors to protect volumes defined as critical structures. A relevant change in the treatment planning is the ability to generate a single isocenter composed of different beam diameters. Such a composite shot design allows an optimized dose distribution shape for each individual shot.

FIG. 4 illustrates a dose calculation interface 40 for a dose engine configured to generate a dose kernel employed that can be employed to generate a radiation treatment plan for a Leksell Gamma Knife® Perfexion™ system. The dose calculation interface 40 can also be employed to simulate what a distribution would look like for a single shot of the Leksell Gamma Knife® Perfexion™ system. A user of the dose calculation interface 40 would follow the steps illustrated in FIG. 4, using the modular dose kernel that defines a dose distribution of a single shot of radiation in an isocenter, to generate a treatment plan. First, the user will set the skull measurements by selecting a “Skull Measurements” selection button. The button will cause a skull measurements setting window 50 to display, as illustrated in FIG. 5. The user can then input the skull measurements in the skull measurements setting window 50, and select the “Save Measurements” button to save a patient's skull measurements. The skull measurements are measured from the skull to the halo or head holder that may be used in the radiation treatment. Since the distribution bone in the skull will affect the dose received by the lesion, these measurements may be used to most accurately plan the radiation treatment.

The user of a Gamma Knife Perfexion can then choose sector sizes in a sector size window 42 by selecting a collimator size of 4, 8, or 16 for each sector or by clicking on one of the selection buttons “All 4 mm”, “All 8 mm” or “All 16 mm” to select a same size collimator for each sector. The user can then select one of the two dose resolution button (0.25 mm/px or 0.5 mm/px) that define the size of the dose per pixels. Selecting of the “Calculate Dose Distribution” button results in the generation of a resultant dose kernel, which displays a simulation of a dose shot distribution in a dose display map region 44 of the dose calculation interface 40. A user can modify the iso-levels and isodoses in a recalculation region 46 of the dose calculation interface 40, and recalculate the resultant dose kernel by selecting a “recalculate” button until the user is satisfied with the resultant dose kernel. FIG. 6a illustrates the dose calculation interface 40 being configured to generate an “All 16 mm” same size collimator for each sector. FIG. 6b illustrates the dose calculation interface 40 being configured to generate a hybrid shots in which collimator sizes of 0, 4, 8 and 16 for different sectors. Different options for beam size could be made available through the treatment planning interface for non-Gamma Knife Perfexion devices.

FIG. 7 illustrates an example embodiment of a method 60 for generating a radiation treatment plan. The method 60 employs the improved Tomosurgery treatment planner. The method begins at 62 where Tomosurgery inputs are loaded into the Tomosurgery treatment planner. The Tomosurgery inputs can include a dose kernel that is either a generic dose kernel from vendor specification or 3^{rd} party calculation for a given treatment delivery apparatus. Alternatively, the dose kernel can be provided by a dose calculation engine, for example, for use with the Leksell Gamma Knife® Perfexion system, as previously discussed. The Tomosurgery inputs can also include a DICOM image set and a DICOM-RT structure set, as previously described in FIG. 1. Optionally, a further input can include a user-defined DICOM dose file. Referring again to FIG. 7, after the Tomosurgery inputs are loaded, the methodology proceeds to Tomosurgery parameter selection at 64. Parameter selection can include selecting shot and slice spacing, desired prescription dose, allowable non lesion dose and critical structure dose and other parameters.

At 66, phase 1 slice optimization is initiated. Phase 1 slice optimization can include dividing a lesion into slices, calculating a raster path, processing those slices in parallel via parallel processing and generating shot path coordinates. After execution of phase 1 slice optimization, a user can view and evaluate preliminary slice plans at 68. The user can then elect to proceed to phase 2 plan optimization at 68. Alternatively, the user can elect to not proceed to phase 2 plan optimization and adjust the Tomosurgery parameters by returning to parameter selection at 64 and/or adjusting the user-defined dose at 72 via a user-defined dose inhomogeneity interface at 74. The user can then return to re-execute phase 1 slice optimization at 66. Once the user is satisfied with the slice optimization results, the user can then elect to execute phase 2 plan optimization at 76. Executing of phase 2 plan optimization can include assembling slice plans, comparing the plan coverage to a desired dose and weighting the various slice plans accordingly. The method then provides a complete dose calculation plan at 78. The complete dose calculation plan 78 can be calculated to view treatment plan cover 80, viewed in a Dose Map with Isodose lines 82, saved 84 and/or exported 86 to be utilized by a treatment delivery apparatus. If the plan is unsatisfactory to the user, the user can return to 72 adjusting the user-defined dose via the user-defined dose inhomogeneity interface at 74.

FIGS. 8a -8i illustrate a visual walkthrough of the treatment planner GUI software during execution of a treatment plan by the treatment planner. At FIG. 8a, a user selects: File->Load Dose Kernel . . . -> to choose a pre-loaded kernel or upload a custom kernel. (NOTE: When properly loaded, a “DOSE:” entry will appear in the small black “Loaded Data” window). At FIG. 8b, a user selects: File ->Load Structure . . . -> to load a structure file. There are two options for loading a structure file. Either select a single ‘RTSS.dcm’ DICOM file containing the structure set, or simultaneously select a text file (containing raw binary volume data) along with a header file (using CTRL+click for multiple selections). FIG. 8c illustrates a structure GUI view. When loaded, the default view is the “Lesion” view. Another view can be selected by choosing a radio button (see yellow box). Critical structures are shown in red. Moves through the z-slices of the structure can be performed using the slider bar (green). Relevant position information is shown in the upper right hand side of the viewbox.

FIG. 8d shows a GUI for choosing initial plan parameters. Values can be adjusted using the 4 sliders in the bottom-right (yellow box above), if desired. Note that defaults are loaded depending on kernel choice. FIG. 8e shows a GUI for viewing initial plan & optimizing. Treatment slices are shown in cyan when planning is complete, with red points indicating intended shot placement (see red outlines). Treatment slice summary is shown in the “Slices” list box (yellow outline). A user can navigate slices using viewbox slider or by clicking on the slice in the list. A user can adjust raster line placement using the “Shift Up”/“Shift Down” buttons, and shot spacing by re-tuning the parameter sliders and clicking “Plan” again (green outlines). A user can click “Optimize” when ready.

FIG. 8f shows a GUI that provides a dose preview, while still calculating weights. Initial shot doses for each treatment slice are shown in cyan, but plan calculation is still ongoing in the background. FIG. 8g shows a GUI for viewing the entire dosespace. A hotspot is shown. Once calculation is completed, a user can click the 3^{rd} tab “DoseSpace” (red). Then the user can click “Plan Dose” to view the calculated dose distribution (blue), and navigate through slices with slider (yellow). FIG. 8h shows a GUI that provides a view of Isodose coverage. A user can Click on “Show Isodose” to see the specified isodose value (relative to maximum specified dose) as a filled red overlay on the dose distribution. (NOTE: Although the weightings are normalized, the final dosespace values can often exceed 1.0 due to additive dose. In this version of the software, one can click on “Normalized” to see the 50% relative to the max recorded dose.) FIG. 8i shows a GUI for running an analysis. A user can click on the “Analysis” tab at the bottom of the screen (yellow outline). Then click “Run Analysis” (red outline) to calculate volume counts and conformity indices. A user can view final shot coordinates and weightings by clicking on a treatment slice in the “Slices” viewbox as before, and examine the list of shots (in the red-text “Shots” box on the right). Finally, a user can click “Save Plan” or “Export Shots” if plan is acceptable (blue outline).

For analyzing existing DICOM-RT Dose Files, a user can load a DICOM-RT structure set file (typically “RTSS.dcm”) or text/header file combination, as in FIG. 8b. A dose kernel is not necessary. A user can click on the “Analysis” tab (as in FIG. 8i), Click the “Load . . . ” button on the left hand side of the window, and choose the RT dose file (typically named “RTDOSE00000000XX.dcm” where XX is a number). When loaded, the cyan textbox on the right hand side of the screen will say “DICOM dose loaded successfully”. A user can then click the “Run DICOM Analysis” button (located below the “Run Analysis” button, shown in FIG. 8i). For viewing the DICOM Image Sets, a user can select File->Load DICOM Image Set, as shown in FIG. 8a. Using CTRL+click or Shift +click, a user can select the range of DICOM files representing the slice-based image set, and wait for processing to complete. When ready, the user can click the “DICOM” tab to view image set, and move through images using the viewbox slider.

FIG. 9 illustrates a user-defined Dose inhomogeneity interface that allows for a user to provide a user-defined adjusted inhomogeneity dose. For example, the user can employ a computer tool such as a paintbrush and adjust priority on the various structures by painting different colors and/or erasing different portions slice-by-slice. By adjusting the priority at different spots, the user can change the homogeneity of the dose at different areas (e.g., create “hot” or “cold” spots). The lesion is shown in black and can be given a high priority, such as a 1.0. The gray structure are considered critical structure and can be given a low priority, such as a 0.1, while the white area is normal tissue and can be given a low to medium priority, such as a 0.2 or 0.3. This allows for a user to redefine dose distribution to specific areas of the structures slice-by-slice and then re-execute the phase I optimization based on the redefined dose distributions.

FIG. 10 illustrates a method 120 for phase 1 slice optimization in accordance with one or more embodiments of the invention. The method begins at 122 where a lesion image is separated according to desired shot sizes and lesion geometry. Each 3D lesion slice is then compress into a single 2D matrix or a slice projection to allow for computationally simpler processing. At 124, each slice is processed separately with two or more slices being processed in parallel with a multi-core processor. At 126, a spacing algorithm computes the best positions for the raster lines to provide the best coverage with the least risk of extraneous dose, and the lesion boundaries along each raster path are determined. The methodology then proceeds to 128. At 128, the coordinates for shots are spaced along each raster path depending on the chosen shot spacing. Shots are assigned the same initial weight for uniform coverage, which will be optimized in phase 2 to eliminate non-uniform dose distributions due to extraneous dose, beam crossover, etc. The methodology then proceeds to 130, to execute a cost function that determines the error with each iteration, and the error is used to adjust the weight for the next iteration, such that weights for each shot are incrementally adjusted based on the error, and then reevaluated. The cycle proceeds until the difference is below a specified threshold.

FIG. 11 illustrates a method 140 for phase 2 plan optimization in accordance with one or more embodiments of the invention. The method begins at 142 where an additional weighting algorithm globally weights each slice in relation to the other slices for 3-dimensional optimization. At 144, the real 3-dimensional dose matrix is populated as well as a desired dose matrix, as opposed to utilizing the compressed version of slices employed in the phase 1 optimization. At 146, an iterative cost function is executed to determine the weight of each slice, until the error is below a predetermined threshold. The iterative cost function compares the actual determined dose to the desired dose based on the slice weights and dose matrix.

FIG. 12 is a schematic block diagram illustrating an example system 600 of hardware components capable of implementing examples disclosed in FIGS. 1-11. The system 600 can include various systems and subsystems. The system 600 can be a personal computer, a laptop computer, a workstation, a computer system, an appliance, an application-specific integrated circuit (ASIC), a server, a server blade center, a server farm, a mobile device, such as a smart phone, a personal digital assistant, an interactive television set, an Internet appliance, portions of a printer, etc.

The system 600 can include a system bus 602, parallel processing units 604, a system memory 606, memory device 608, the improved Tomosurgery treatment planner 610 residing in memory or a logic device some of which is discussed in paragraphs 27-30 above, a communication interface 612 (e.g., a network interface), a communication link 614, a display 616 (e.g., a video screen), and an input device 618 (e.g., a keyboard and/or a mouse). The system bus 602 can be in communication with the parallel processing units 604 and the system memory 606. The additional memory device 608, such as a hard disk drive, server, stand alone database, or other non-volatile memory and the improved Tomosurgery treatment planner 610, can also be in communication with the system bus 602. The system bus 602 operably interconnects the parallel processing units 604, the memory devices 606 and 608, the improved Tomosurgery treatment planner 610, the communication interface 612, the display 616, and the input device 618. In some examples, the system bus 602 also operably interconnects an additional port (not shown), such as a universal serial bus (USB) port.

The parallel processing units 604 can be a multi-core processing device that can execute task in parallel, such as processing and computing various tasks executed by the improved Tomosurgery treatment planner. The parallel processing units 604 execute a set of instructions to implement the operations of examples disclosed herein.

The additional memory devices 606 and 608 can store data, programs, instructions, database queries in text or compiled form, and any other information that can be needed to operate a computer. The memories 606 and 608 and the improved Tomosurgery treatment planner 610 can be implemented as computer-readable media (integrated or removable) such as a memory card, disc drive, compact disk (CD), or server accessible over a network. In certain examples, the memories 606 and 608 can comprise text, images, video, and/or audio.

In operation, the system 600 can be used to implement, for example, the improved Tomosurgery treatment planner 610. Machine (e.g., computer) executable logic for implementing the system can reside in the system memory 606, and/or in the memory devices 608 and/or Tomosurgery treatment planner 610 in accordance with certain examples. The parallel processing units 604 executes machine readable instructions originating from the system memory 606 and the memory devices 608 and Tomosurgery treatment planner 610.

Where the disclosure or claims recite “a,” “an,” “a first,” or “another” element, or the equivalent thereof, it should be interpreted to include one or more than one such element, neither requiring nor excluding two or more such elements. Furthermore, what have been described above are examples. It is, of course, not possible to describe every conceivable combination of components or methods, but one of ordinary skill in the art will recognize that many further combinations and permutations are possible. Accordingly, the invention is intended to embrace all such alterations, modifications, and variations that fall within the scope of this application, including the appended claims.

Having described the invention the following is claimed:

1. A computer-implemented method for generating a radiation treatment plan, the method comprising:
reading a modular radiation dose kernel that defines a three dimensional radiation isocenter dose formed from a sum of a plurality of radiation beams;
reading data that defines a three dimensional closed surface representing a lesion; and
generating a radiation treatment plan comprising:
determining a three dimensional radiation isocenter dose from the loaded radiation dose kernel;
logically dividing the three dimensional closed surface representing a biological object such as a whole organ, a lesion, a critical structure, a tissue, or a population of cells, into a plurality of treatment slices;
determining and optimizing an initial path plan for moving the three dimensional radiation isocenter dose through each treatment slice for each of the treatment slices; and
determining and optimizing a final path plan for moving the three dimensional radiation isocenter dose through all of the treatment slices in order to deliver an optimally conformal distribution of the desired dose mitigating beam crossing effects that occur in/between treatment slices during the initial path plan.

2. The method of claim 1, wherein the initial and/or final path plans include determining dose rates at different locations by modulating the speed of the dose during the moving of the three dimensional radiation isocenter dose.

3. The method of claim 1, wherein the optimizing the initial and/or final path plans include determining the weight and priority of the dose at different locations utilizing one or more cost functions.

4. The method of claim 1, wherein the determining the initial and/or final plans include determining on and off times of the dose during the moving to provide discrete doses that simulate a continuously moving dose.

5. The method of claim 1, wherein the determining and/or optimizing the initial and/or final path plans are executed employing single or parallel processing utilizing one of more of central processing units (CPUs) or multicore central processing units and/or single or multithreaded parallel processing units and/or graphical processing units (GPUs).

6. The method of claim 1, further comprising displaying a calculated, optimized treatment plan that may incorporate user-selected parameters, or utilize a set of baseline parameters, via a user interface that displays the one or more results associated with the planning, and allows a user to change parameters and make changes to the defined dose distribution for at least one of the plurality of slices.

7. The method of claim 6, further comprising receiving changes to the defined dose distribution for one or more treatment slices from treatment planning user interface and instructions to re-execute the initial and/or final path plan.

8. The method of claim 1, wherein the radiation dose kernel is generated by a dose engine via a dose calculation interface that allows a user to define collimator sizes over one or more sectors.
